(19) United States
(12) Patent Application Publication
(10) Pub. No.: US 2019/0110748 A1
(43) Pub. Date: Apr. 18, 2019

(54) SMART GARMENT INTEGRATED WITH ELECTRIC WIRES COATED WITH CONDUCTIVE SILICONE RUBBER

(71) Applicant: WAVE COMPANY CO., LTD., Seoul (KR)

(72) Inventors: Na-Yun Cho, Seoul (KR); Sang-Chul Lee, Seoul (KR)

(21) Appl. No.: 16/199,537

(22) Filed: Nov. 26, 2018

Related U.S. Application Data
(63) Continuation of application No. PCT/KR2017/014103, filed on Dec. 4, 2017.

(30) Foreign Application Priority Data
Sep. 12, 2017 (KR) 10-2017-0116867

(51) Int. Cl. A61B 5/00; A61B 5/11; A41D 1/00

(52) U.S. Cl. CPC A61B 5/6804; A41D 1/002; A61B 5/1114; A61B 5/1126

(57) ABSTRACT

A smart garment configured to easily attach and detach sensors and devices to the smart garment for easy washing. The smart garment includes: a garment fabric; a plurality of electric wires formed by coating line patterns on an outer surface of the garment fabric, the electric wires including electrodes on both ends thereof; a sensor and a measuring device electrically connected to ends of the electric wires; and a controller electrically connected to the other ends of the electric wires, wherein the electric wires are formed by coating the outer surface of the garment fabric with a liquid silicone resin mixed with conductive powder according to the line patterns and curing the silicone resin, and input/output terminals of the sensor, the measuring device, and the controller are detachably coupled to the electrodes of the electric wires.

### REFERENCE TO RELATED APPLICATIONS

This is a continuation of International Patent Application PCT/KR2017/014103 filed on Dec. 4, 2017, which designates the United States and claims priority of Korean Patent Application No. 10-2017-0116867 filed on Sep. 12, 2017, the entire contents of which are incorporated herein by reference.

### FIELD OF THE INVENTION

The present invention relates to a smart garment, and more particularly, to a smart garment which is configured to easily attach and detach sensors and devices to and from the smart garment for easy washing and is provided with electric wires coated with flexible and conductive silicone rubber for good activity.

### BACKGROUND OF THE INVENTION

Motion recognition is a technique used in applications such as games, film production, or motion pattern analysis, and according to the motion recognition technique, a person wearing a garment to which various sensors are attached may play games or participate in movie filming or an exercise tolerance test.

A smart garment having a bio-signal measuring function is disclosed in Korean Patent Application Laid-open No. 10-2008-0021267. The disclosed smart garment includes a garment made of a material that can be in tight contact with the body of a user and a bio-signal measuring module coupled to an inner surface of the garment to detect bio-signals generated from the body of the user, wherein the bio-signal measuring module includes a sensor including a conductive fiber, a PCB block including an electric circuit block provided on a PCB for driving the sensor and storing bio-signals received from the sensor, and a connection member formed of a conductive fiber and having a given length for coupling the PCB and the sensor to each other, wherein an accommodation means is provided on the inner surface of the garment to accommodate the PCB block.

In the above-described structure, however, it is difficult and complex to attach the sensor and a control device to the smart garment and wash the smart garment.

Moreover, although small devices such as devices for measuring a pulse rate and an oxygen saturation degree are available in the market in addition to sensors, such devices are not attached to the garment, and thus it is inconvenient to separately carry such devices.

### SUMMARY OF THE INVENTION

Therefore, an object of the present invention is to provide a smart garment configured to easily attach and detach various devices such as sensors to and from the smart garment.

Another object of the present invention is to provide a smart garment provided with coated electrodes for easy washing.

To accomplish the above objects, a smart garment includes: a garment fabric; a plurality of electric wires formed by coating line patterns on an outer surface of the garment fabric, the electric wires including electrodes on both ends thereof; a sensor and a measuring device electrically connected to ends of the electric wires; and a controller electrically connected to the other ends of the electric wires, wherein the electric wires are formed by coating the outer surface of the garment fabric with a liquid silicone resin mixed with conductive powder according to the line patterns and curing the silicone resin, and input/output terminals of the sensor, the measuring device, and the controller are detachably coupled to the electrodes of the electric wires.

Preferably, the input/output terminals and the electrodes may be electrically conductive Velcro (hook & loop tape) buttons or metal snap buttons.

Preferably, a penetration hole may be formed in the garment fabric at a position to which the measuring device is attached, and an adhesive silicone coating layer may be formed on a lower surface of the garment fabric around a peripheral portion of the penetration hole or on a surface of the measuring device such that the adhesive silicone coating layer may serve as a spacer or fix the garment fabric to the skin.

Preferably, the sensor, the measuring device, or the controller may be cover with a cover having air permeability, and the cover may be detachably attached to the garment fabric.

Preferably, the controller may include: a chargeable lithium-ion battery to receive power from the lithium-ion battery; and a Bluetooth module to transmit detection signals received from the sensor and the measuring device to an external device by Bluetooth communication.

According to the structures, since the electric wires are formed by directly coating the garment fabric with an electrically conductive silicone rubber layer, intense activities such as exercise may be possible owing to the durability, elasticity, and flexibility of silicone rubber.

In addition, the input/output terminals of the sensor, the measuring device having a tiny size, and the controller are formed of electrically conductive Velcro or metal snap buttons, and corresponding terminals of the electric wires are also formed of electrically conductive Velcro or metal snap buttons. Thus, the sensor, the tiny measuring device, and the controller may be easily attached to and detached from the garment fabric. As a result, the sensor, the tiny measuring device, and the controller may be simply detached from the smart garment, and then only the smart garment may be easily washed and managed.

Since all the sensor, the tiny measuring device, and the controller can be attached to the garment fabric, various pieces of bio-information may be obtained at a time, and bio-information may be collected in real time during exercise or work.

### BRIEF DESCRIPTION OF THE DRAWINGS

FIG. 1 illustrates an example of a smart garment according to the present invention.

FIG. 2 illustrates how a sensor is attached to a garment fabric.

FIG. 3 illustrates how a measuring device is attached to the garment fabric.

FIG. 4 illustrates how a controller is attached to the garment fabric.

### DETAILED DESCRIPTION OF THE INVENTION

Technical terms used in the present invention are only for explaining specific embodiments while not limiting the present invention. In addition, unless otherwise defined, technical terms used in the present invention have the same meaning as commonly understood by those of ordinary skill in the art and will not be interpreted in an overly broad or narrow sense. In addition, if technical terms used in the present invention are incorrect to exactly express the idea of the present invention, the technical terms should be interpreted as terms by which those of ordinary skill in the art can correctly understand the idea of the present invention. In addition, general terms used in the present invention may be interpreted as defined in dictionaries or according to the contextual meanings, and should not be interpreted in an overly narrow sense.

Hereinafter, specific embodiments of the present invention will be described in detail with reference to the accompanying drawings.

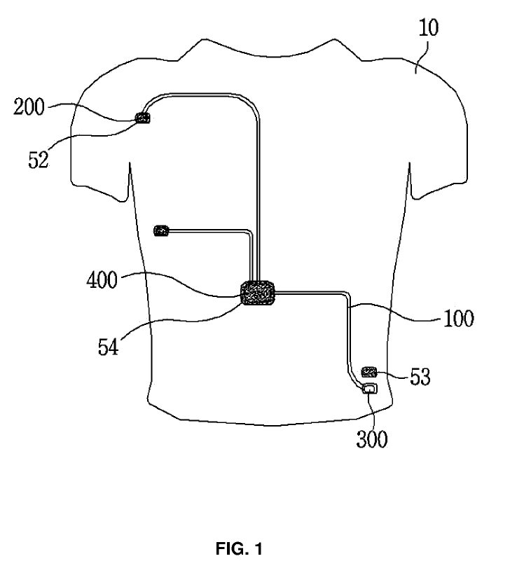

FIG. 1 illustrates an example of a smart garment according to the present invention.

A plurality of electric wires 100 extend in line patterns from a controller 400 attached to an outer surface of a garment fabric 20 such as a T-shirt fabric, and a sensor 200 and a measuring device 300 are detachably attached to electrodes formed on end portions of the electric wires 100.

Herein, for example, the sensor 200 may be a motion recognition sensor, the measuring device 300 may be a device for measuring a pulse rate or an oxygen saturation, and the controller 400 is a device for receiving signals from the sensor 200 and the measuring device 300 and wirelessly transmitting the signals to a remote place.

The electric wires 100 may be formed, for example, by coating the garment fabric 20 with a liquid electrically conductive resin and curing the liquid electrically conductive resin. In the example, each of the sensor 200 and the measuring device 300 is connected to the controller 400 through a single wire 100. However, the number of electric wires 100 corresponds to input/output terminals of the sensor 200, the measuring device 300, and the controller 400.

The sensor 200, the measuring device 300, and the controller 400 may be covered with covers 52, 53, and 54, and FIG. 1 illustrates the cover 53 separated from the measuring device 300.

The covers 52, 53, and 54 are for preventing detachment and separation of the sensor 200, the measuring device 300, and the controller 400 and protecting the sensor 200, the measuring device 300, and the controller 400 from surroundings when a person wearing the garment works out. The covers 52, 53, and 54 may be formed of a material having high air permeability and may be attached using Velcro (hook & loop tape).

According to this structure, all the sensor 200, the measuring device 300, and the controller 400 may be attached to the garment fabric 20, and thus there are advantages in that various pieces of bio-information can be obtained at a time, and bio-information can be collected in real time during exercise or work.

Hereinafter, attachment and detachment of each component will be described in detail.

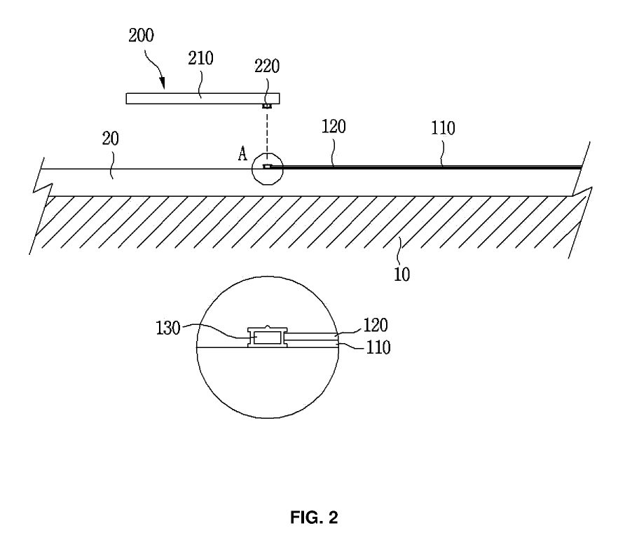

FIG. 2 illustrates how the sensor 200 is attached to the garment fabric 20.

The sensor 200, for example, a motion recognition sensor, may include a main body 210 and at least one terminal 220, and the terminal 220 may be an electrically conductive Velcro or snap button.

An electric wire 100 is coated on the garment fabric 20 covering the skin 10 of a person's body, and at least one electrode 130 is formed on an end portion of the electric wire 100. The electrode 130 may be an electrically conductive Velcro or metal snap button corresponding to the terminal 220 of the sensor 200.

The electric wire 100 may include an electrically conductive silicone rubber layer 110 and an insulative silicone rubber layer 120. Alternatively, the electric wire 100 may be formed of a single silicone rubber layer which is electrically conductive in the inside thereof and not electrically conductive on the surface thereof, or the surface of the electric wire 100 may be insulated by spray coating, painting, or bonding a film instead of providing the insulative silicone rubber layer 120.

The electrically conductive silicone rubber layer 110 is formed by mixing conductive powder with a silicone resin being a binder, and since the electrically conductive silicone rubber layer 110 has high electrical conductivity and high adhesion to the garment fabric 20, the electrically conductive silicone rubber layer 110 may be durable even when the garment fabric 20 is rubbed and bent.

Therefore, owing to high adhesion, elasticity, and flexibility of the electrically conductive silicone rubber layer 110 of the electric wire 100, the garment may be easily washed and may not be damaged after being washed.

In addition, the insulative silicone rubber layer 120 prevents electric shock when power is applied to the electrically conductive silicone rubber layer 110 and has adhesion and elasticity owing to a silicone binder. Therefore, the insulative silicone rubber layer 120 further guarantees durability against external influences such as washing.

In this case, the insulative silicone rubber layer 120 may completely surround the electrically conductive silicone rubber layer 110, and the electrode 130 formed on an end portion of the electrically conductive silicone rubber layer 110 is exposed to the outside.

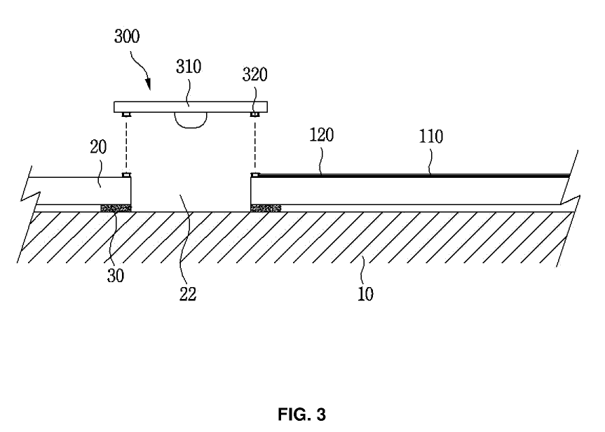

FIG. 3 illustrates how the measuring device 300 is attached to the garment fabric 20.

The measuring device 300, for example, a tiny device for measuring a pulse rate/oxygen saturation, may include a main body 310 and at least one terminal 320, and the terminal 320 may be an electrically conductive Velcro or metal snap button.

An electric wire 100 is coated on the garment fabric 20 covering the skin 10 of a person's body, and at least one electrode 130 is formed on an end portion of the electric wire 100. The electrode 130 may be an electrically conductive Velcro or metal snap button corresponding to the terminal 320 of the measuring device 300.

Unlike the case of attaching the sensor 200, a penetration hole 22 is formed in the garment fabric 20 at a position to which the measuring device 300 will be attached, and an adhesive silicone coating layer 30 is formed along a peripheral portion of the penetration hole 22 between the garment fabric 20 and the skin 10. The adhesive silicone coating layer 30 functions as a spacer and fixes the garment fabric 20 to the skin 10. In addition, during measurement with the measuring device 300, the adhesive silicone coating layer 30 prevents a measuring part from wobbling and thus guarantees more precise measurement.

Instead of forming the adhesive silicone coating layer 30 on the garment, the adhesive silicone coating layer 30 may be formed on a particular portion of the measuring device 300 for the same purposes.

Thus, the measuring device 300 may be spaced apart from the skin 10 by a given distance, and for example, an optical measurement may be possible through the penetration hole 22 by using the measuring device 300.

Attachment, detachment, and related structures of the measuring device 300 are the same as those of the sensor 200, and thus detailed descriptions thereof will not be repeated here.

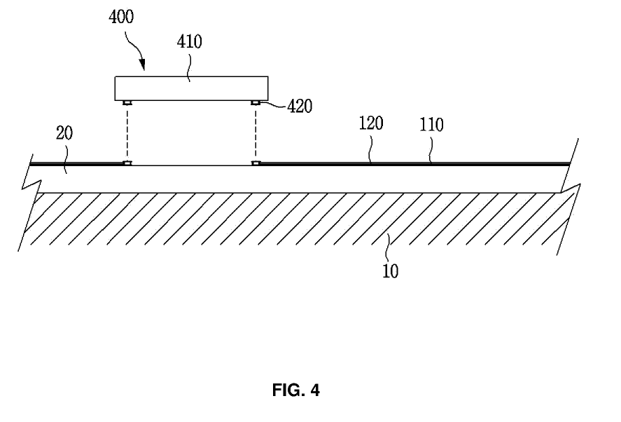

FIG. 4 illustrates how the controller 400 is attached to the garment fabric 20.

The controller 400 includes a main body 410 and a plurality of terminals 420 and 421 provided on the main body 410 for electrical connection with the sensor 200 and the measuring device 300.

The controller 400 may include a chargeable lithium-ion battery to receive power from the lithium-ion battery and may include a Bluetooth module to transmit detection signals received from the sensor 200 and the measuring device 300 to an external device such as a cellar phone by Bluetooth communication.

Besides the Bluetooth module, the controller 400 may include a short-range wireless communication module such as an NFC or RF module for short-range wireless communication.

As described above, the smart garment of the present invention has many advantages.

Since the electric wires are formed by directly coating the garment fabric with a silicone rubber layer, the smart garment may be conveniently managed and washed owing to high adhesion, elasticity, and flexibility of the silicone rubber layer.

In addition, the input/output terminals of the sensor, the tiny measuring device, and the controller are formed of electrically conductive Velcro or metal snap buttons, and corresponding terminals of the electric wires are also formed of electrically conductive Velcro or metal snap buttons. Thus, the sensor, the tiny measuring device, and the controller may be easily attached to and detached from the garment fabric. As a result, the sensor, the tiny measuring device, and the controller may be simply detached from the garment fabric, and then only the garment fabric may be washed.

In addition, since all the sensor, the tiny measuring device, and the controller can be attached to the garment fabric, various pieces of bio-information may be obtained at a time, and bio-information may be collected in real time during exercise or work.

Those of ordinary skill in the art may make changes or modifications from the above description without departing from the spirit and scope of the present invention. The embodiments of the present invention are for illustrative purposes only and are not intended to limit the scope of the present invention. Therefore, the scope of the present invention should be construed according to the appended claims, and it should be understood that all technical ideas equivalent to those described above are within the scope of the present invention.

What is claimed is:

1. A smart garment providing high activity and comprising:
a garment fabric;
a plurality of electrically conductive patterns coated on an outer surface of the garment fabric and insulated by insulative silicone rubber surrounding the conductive patterns, both ends of the conductive patterns being provided with electrodes;
a sensor and a measuring device electrically connected to electrodes provided on ends of the conductive patterns; and
a controller electrically connected to electrodes provided on the other ends of the conductive patterns,
wherein the conductive patterns are formed by coating the outer surface of the garment fabric with a liquid silicone resin mixed with electrically conductive powder according to predetermined lines and curing the silicone resin, and
input/output terminals of the sensor, the measuring device, and the controller are detachably coupled to the electrodes of the conductive patterns.

2. The smart garment of claim 1, wherein the input/output terminals and the electrodes are metal snap buttons.

3. The smart garment of claim 1, wherein the sensor, the measuring device, or the controller is cover with a cover having air permeability, and
the cover is detachably attached to the garment fabric.

4. A smart garment providing high activity and comprising:
a garment fabric;
a plurality of electrically conductive patterns coated on an outer surface of the garment fabric and insulated by insulative silicone rubber surrounding the conductive patterns, both ends of the conductive patterns being provided with electrodes; and
a measuring device electrically connected to an electrode provided on an end of the conductive patterns,
wherein the conductive patterns are formed by coating the outer surface of the garment fabric with a liquid silicone resin mixed with electrically conductive powder according to predetermined lines and curing the silicone resin,
an input/output terminal of the measuring device is detachably coupled to the electrode of the conductive patterns, and
a penetration hole is formed in the garment fabric at a position to which the measuring device is attached, and an adhesive silicone coating layer is formed on a lower surface of the garment fabric around a peripheral portion of the penetration hole so as to be attached to the skin, such that a gap is formed between the measuring device and the skin, and the garment fabric is fixed to the skin.
